(19) United States
(12) Patent Application Publication
(10) Pub. No.: US 2020/0315648 A1
(43) Pub. Date: Oct. 8, 2020

(54) TUBULAR TISSUE REMOVAL PROCEDURE

(71) Applicant: Covidien LP, Mansfield, MA (US)

(72) Inventor: Robert H. Knapp, Middlebury, CT (US)

(21) Appl. No.: 16/809,051

(22) Filed: Mar. 4, 2020

Related U.S. Application Data
(60) Provisional application No. 62/830,865, filed on Apr. 8, 2019.

(51) Int. Cl. A61B 17/3207; A61B 17/115

(52) U.S. Cl. CPC A61B 17/3207; A61B 2017/12004; A61B 2017/1157; A61B 17/1155

(57) ABSTRACT

A method for removing diseased tissue from a tubular organ includes receiving a distal end portion of a tubular body of a surgical stapling device within a tubular organ of a patient. The method further includes retracting a diseased section of tissue of the tubular organ within a lumen of the tubular body. Proximal and distal margin lines of the tubular organ are then positioned adjacent a cylindrical anvil member disposed on the distal end portion of the tubular body. A cartridge assembly is then received within the tubular body in alignment with the anvil member such that sections of healthy tissue are disposed between the cartridge assembly and the anvil assembly. A plurality of staples is then ejected radially outward from within the cartridge assembly through the sections of healthy tissue disposed between the cartridge assembly and the anvil member, and into the anvil member. The diseased tissue is removed through the tubular body of the surgical stapling device.

### CROSS-REFERENCE TO RELATED APPLICATIONS

This application claims the benefit of and priority to U.S. Provisional Patent Application Ser. No. 62/830,865 filed Apr. 8, 2019, the entire disclosure of which is incorporated by reference herein.

### BACKGROUND

### Technical Field

The present disclosure relates generally to methods of removing sections of diseased tissue from tubular organs, and more specifically, to methods for removing sections of diseased tissue from tubular organs using intussusception.

### Background of Related Art

Diseased gastrointestinal tissue sometimes must be removed, such as in cases of diverticulitis, cancer, and ischemic bowel. The procedure may be completed laparoscopically using surgical staplers to transect out the unhealthy tissue and seal the tissue shut to prevent leakage of luminal content into the abdominal cavity and to provide hemostasis. Before the diseased tissue can be removed from the body, blood vessels and connective tissue to the diseased tissue are cut and sealed. Once, the diseased tissue is separated from the blood vessels, connective tissue, and healthy tissue, it is removed from the body through the abdominal wall. If the specimen is small enough, it can be removed through an abdominal port. However, if the diseased tissue is too large to fit through the abdominal port, a larger incision is required or the procedure must be completed in an open manner.

With reference to FIGS. 1 and 2, in order to restore the gastrointestinal (GI) tract, the oral and aboral ends of where the diseased tissue was removed must be anastomosed either using a suturing technique or an end-to-end anastomosis (EEA) stapler. The EEA stapler is used to staple together two tubular tissue structures using a circular pattern.

Generally, the anastomosis technique requires at least four ports into the abdomen—two five millimeter ports for grasping hand tools/electrocautery, one ten millimeter port for a camera, and one twelve millimeter port for the stapling device. When removing cancerous resected tissue, it is a concern to limit cancerous tissue touching any healthy tissue as it may seed healthy areas with cancerous cells. Additionally, when performing procedures near the pelvic floor in proximity to the anus there is limited space to resect and reattach tissue.

Therefore, it would be beneficial to have a stapling procedure in which all tissue resection and anastomosis is performed from within the target tissue and all diseased tissue would be removed through a natural orifice, (e.g., anus).

### SUMMARY

A method for removing diseased tissue from a tubular organ is disclosed. The method includes receiving a distal end portion of a tubular body of a surgical stapling device within a tubular organ of a patient, retracting a portion of the tubular organ including a diseased section of tissue within a lumen of the tubular body, positioning proximal and distal margin lines of the portion of the tubular organ including the diseased section of tissue adjacent a cylindrical anvil member disposed on the distal end portion of the tubular body, receiving a cartridge assembly within the tubular body in alignment with the cylindrical anvil member such that sections of healthy tissue are disposed between the cartridge assembly and the cylindrical anvil assembly, ejecting a plurality of staples radially outward from within the cartridge assembly through the sections of healthy tissue disposed between the cartridge assembly and the cylindrical anvil member, and into the cylindrical anvil member, and removing the diseased tissue through the tubular body of the surgical stapling device.

In embodiments, the method further includes identifying the proximal and distal margin lines of the tubular organ. The method may include ligating and severing blood vessels and connective tissue attached to the diseased section of tissue. Ligating and severing the blood vessels and connective tissue may include using an electrocautery device.

Ejecting the plurality of staples may include cutting the tissue adjacent the proximal and distal margin lines to separate the diseased section of tissue from the tubular organ. Ejecting the plurality of staples may include receiving a drive member within the cartridge assembly. Ejecting the plurality of staples may include forming first and second staple lines in the section of healthy tissue. In addition, ejecting the plurality of staples may include cutting the section of healthy tissue between the first and second staple lines.

In embodiments, retracting the portion of the tubular organ including a diseased section of tissue within the lumen of the tubular body includes inverting the portion of the tubular organ. Inverting the portion of the tubular organ may include grasping tissue of the tubular organ distal of the section of diseased tissue and pulling the tissue proximally within the tubular body.

### BRIEF DESCRIPTION OF THE DRAWINGS

Embodiments of the presently disclosed methods for removing sections of diseased tissue from tubular organs are described herein with reference to the drawings, wherein:

FIG. 1A is a schematic view of a gastrointestinal (GI) tract illustrating the removal of diseased tissue from a colon, according to the prior art;

FIG. 1B is a schematic view of the GI tract shown in FIG. 1A illustrating anastomosis of the colon, according to the prior art;

FIG. 2A is a schematic view of an anastomosis procedure including an end-to-end anastomosis (EEA) stapler prior to the stapling procedure, according to the prior art;

FIG. 2B is a schematic view of a complete anastomosis from the anastomosis procedure illustrated in FIG. 2A;

FIG. 3A is a schematic view of a tubular organ with an intussesception;

FIG. 3B is an enlarged, partial cross-sectional view of the intussesception shown in FIG. 3A;

FIG. 4 is a schematic view of a tubular organ including a diseased section of tissue;

FIG. 5 is a side view of distal end portions of a tubular body, a cartridge assembly, and a driver member of a surgical stapling instrument suitable for use with the stapling procedure of the present disclosure;

FIG. 6 is a schematic view of the tubular organ shown in FIG. 4 and the distal end of the tubular body shown in FIG. 5 received within the tubular organ;

FIG. 7 is a schematic view of the tubular organ and the distal end of the tubular body shown in FIG. 6, further including a plurality of graspers and electrosurgical devices;

FIG. 8 is a schematic view of tubular organ and distal end of the tubular body shown in FIGS. 6 and 7, further including the distal end of the cartridge assembly shown in FIG. 5 operably disposed within the tubular body;

FIG. 9 is a schematic view of the tubular organ and distal ends of the tubular body and cartridge assembly shown in FIG. 8, further including the distal end of the driver member shown in FIG. 5 operably disposed with the cartridge assembly;

FIG. 10 is a schematic view of the tubular organ and distal end of the tubular body shown in FIGS. 6-9, as the diseased section of tissue is removed through the tubular body; and

FIG. 11 is a schematic view of the tubular organ shown in FIG. 4 with the diseased section of tissue removed.

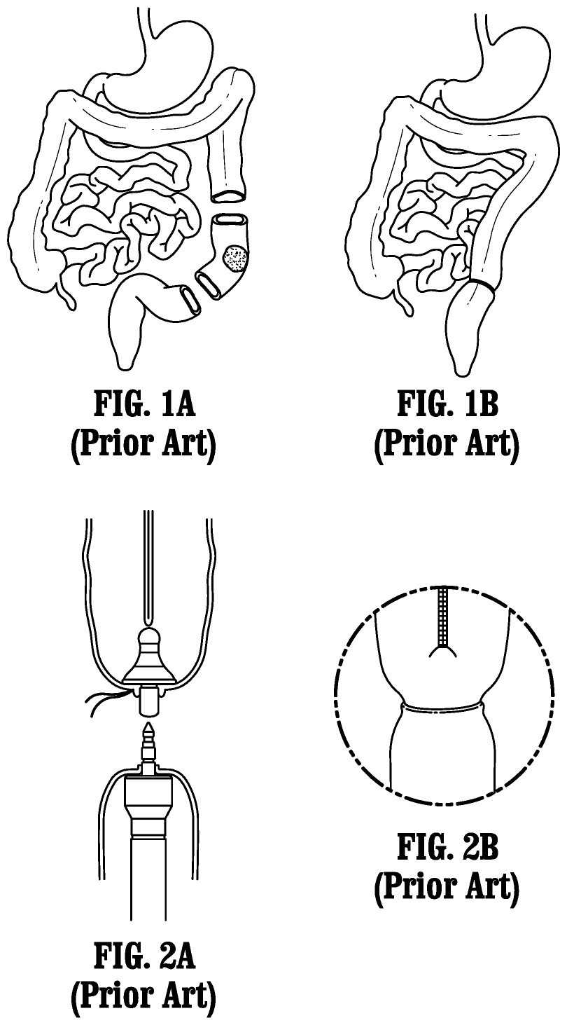

### DETAILED DESCRIPTION OF THE DRAWINGS

Embodiments of the presently disclosed methods for removing sections of diseased tissue from a tubular organ are described in detail with reference to the drawings, wherein like reference numerals designate corresponding elements in each of the several views. In the drawings and the description that follow, the term “proximal” refers to the end of the surgical stapling instrument that is closer to the clinician, whereas the term “distal” refers to the end of the surgical stapling instrument that is farther from the clinician. In addition, the term “clinician” is used generally to refer to medical personnel including doctors, nurses, and support personnel.

The methods of the present disclosure eliminate the need for end-to-end anastomosis (EEA) stapling, and of cutting over previous staple lines. The presently disclosed methods may also require fewer abdominal ports and may allow for better containment of the diseased tissue. In addition, the presently disclosed methods may involve less tissue at the rectal stump which allows for greater margins, and permits removal of diseased tissue relatively close to the pelvic floor, leaving more healthy tissue.

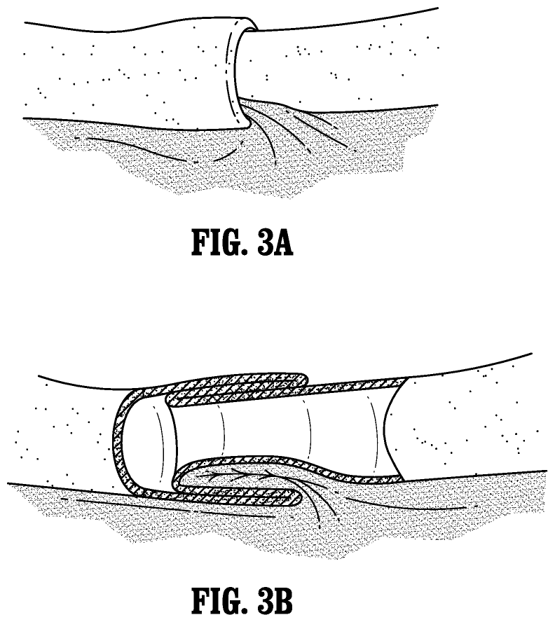

The methods described in the present disclosure utilize the pathology of a medical disorder which is prominent in infants known as intussusception. With reference to FIG. 3, intussusception is when the intestine slides into an adjacent section of the intestine which causes a telescoping effect. When intussusception occurs naturally, the blood supply to the otherwise healthy intestinal tissue can become blocked. However, if intussusception is induced after the bloody supply is removed for the intussuscepted or diseased tissue, the intussuscepted tissue may be stapled, cut, and removed with minimal adverse effects. The intussuscepted tissue may then be removed through a natural orifice (most likely the anus), instead of removing the diseased tissue through the abdominal wall.

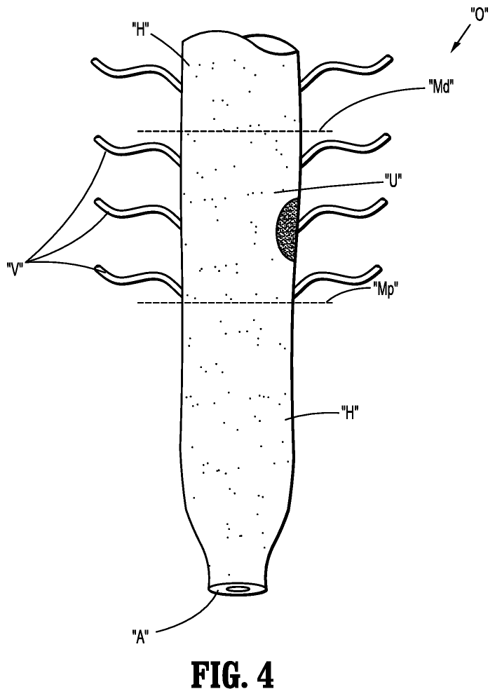

With reference now to FIGS. 4-6, the methods of removing sections of diseased tissue from tubular organs utilizing intussusception of the diseased tissue will be described.

Referring initially to FIG. 4, a section of a tubular organ “0”, e.g., colon, including a healthy tissue “H” and a section of diseased or unhealthy tissue “U”, is shown. The diseased tissue “U” is bound by a proximal margin line “Mp” and a distal margin line “Md”. The proximal and distal margin lines “Mp”, “Md” may be determined by the surgeon based any number of factors including, for example, the amount of diseased tissue and the amount of available healthy tissue. The tubular organ “O” is supplied with blood by a plurality of vessels “V”. In embodiments, and as shown, the tubular organ “O” may be accessed through the anus “A” of the patient.

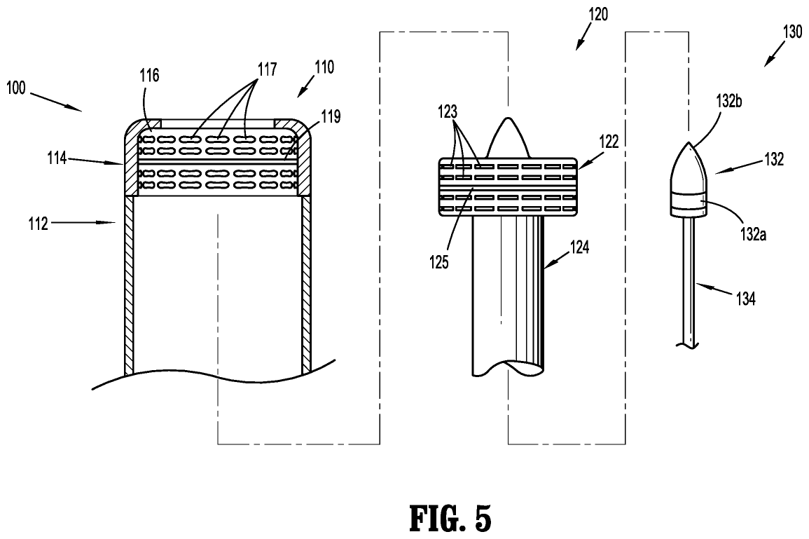

Turning to FIG. 5, a surgical stapling device suitable for completing the tissue removal procedures according to the methods of the present disclosure is shown generally as surgical stapling device 100. The surgical stapling device 100 includes a tubular body 110 having a tapered distal end portion 112. The tapered distal end portion 112 includes a cylindrical anvil member 114. The cylindrical anvil member 114 includes a cylindrical inner surface 116 defining a plurality of staple forming pockets 117 and may define an annular groove 119 for accommodating engagement of a cutting member (not shown).

The surgical stapling device 100 also includes a cartridge assembly 120 having a staple cartridge 122 disposed on a distal end of a tubular member 124. The staple cartridge 122 includes a substantially cylindrical body defining a plurality of staple receiving pockets 123 for supporting a plurality of staples (not shown) and an annular slot 125 for accommodating a cutting member (not shown). The staple cartridge 122 is configured to be received within the cylindrical anvil member 114 of the tubular body 110 and to eject the plurality of staples radially outward therefrom into the staple forming pockets 117 of the cylindrical anvil member 114.

The surgical stapling device 100 further includes a staple driver 130 having staple driving member 132 disposed on a distal end of a drive rod 134. The staple driving member 132 includes a cylindrical body portion 132a and a tapered distal portion 132b. The staple driving member 132 is configured to be received within the staple cartridge 122 and engage staple pushers (not shown) disposed within the staple cartridge 120 to eject the plurality of staples radially outward from within the staple cartridge 122 towards the cylindrical inner surface 116 of the cylindrical anvil member 114.

Although the surgical stapling device 100 is shown and described with an anvil and cartridge configuration in which the plurality of staples are ejected radially outward from the staple cartridge 122 into the cylindrical anvil member 114, it is envisioned that the staple cartridge may instead be disposed outward of the anvil member. In this manner the plurality of staples would be ejected radially inward from the staple cartridge. In embodiments, the stapler driving member may include an annular portion configured to be received about the staple cartridge.

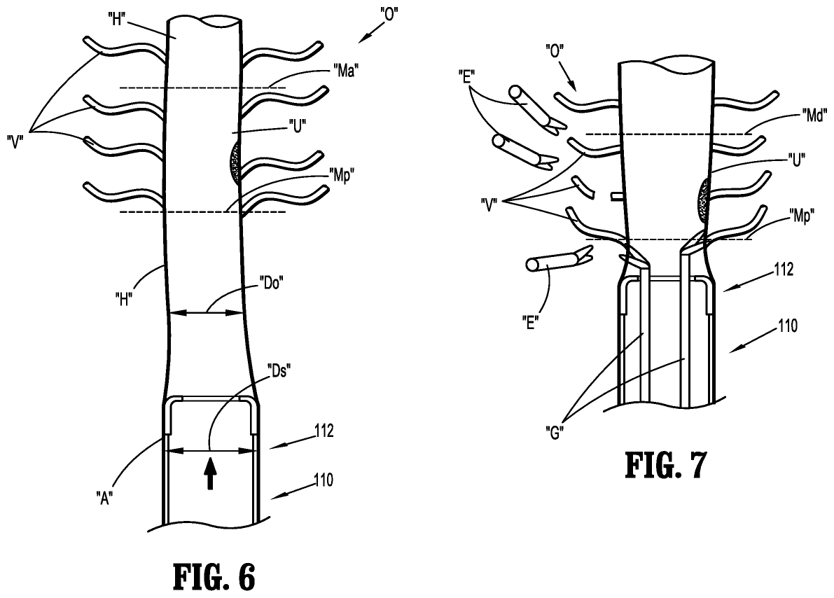

With reference to FIG. 6, during the stapling procedure of the present disclosure, the tapered distal end portion 112 of the tubular body 110 of the surgical stapling device 100 is received through the anus “A” of the patient. In embodiments, the tapered distal end portion 112 of the tubular body 110 of the surgical stapling device 100 includes a diameter “Ds” slightly larger than the diameter “Do” of the tubular organ “O”. Because the diameter “Ds” of the tapered distal end portion 112 is larger than the diameter “Do” of the tubular organ “O”, receipt of the tapered distal end portion 112 of the tubular body 110 within the tubular organ “O” stretches the tissue of the tubular organ “O”, thereby facilitating intussusception of the diseased tissue “U”.

Turning to FIG. 7, the tubular body 110 of the surgical stapling device 100 is inserted through the anus “A” of the patient until the tapered distal end portion 112 of the tubular body 110 is positioned adjacent the proximal margin line “Mp” of the diseased tissue “U”. The surgeon may then use a grasping tool(s) “G” to invert the diseased tissue “U” upon itself and retract the diseased tissue “U” into the tapered distal end portion 112 of the tubular body 110 to cause intussusception of the diseased tissue “U”.

While inverting the diseased tissue “U”, the surgeon may use one or more electrocautery device(s) “E” in the abdominal cavity to ligate or seal the plurality of vessels “V” supplying the diseased tissue “U” and to dissect any connective tissue (not shown) supporting the diseased tissue “U”. Alternatively, the vessels “V” may be tied off using sutures and/or crimped using surgical clips prior to being severed. The plurality of vessels “V” and the connective tissue may be ligated and separated from the diseased tissue “U” prior to inversion and retraction of the diseased tissue “U”, and/or simultaneously with the inversion and retraction of the diseased tissue “U”.

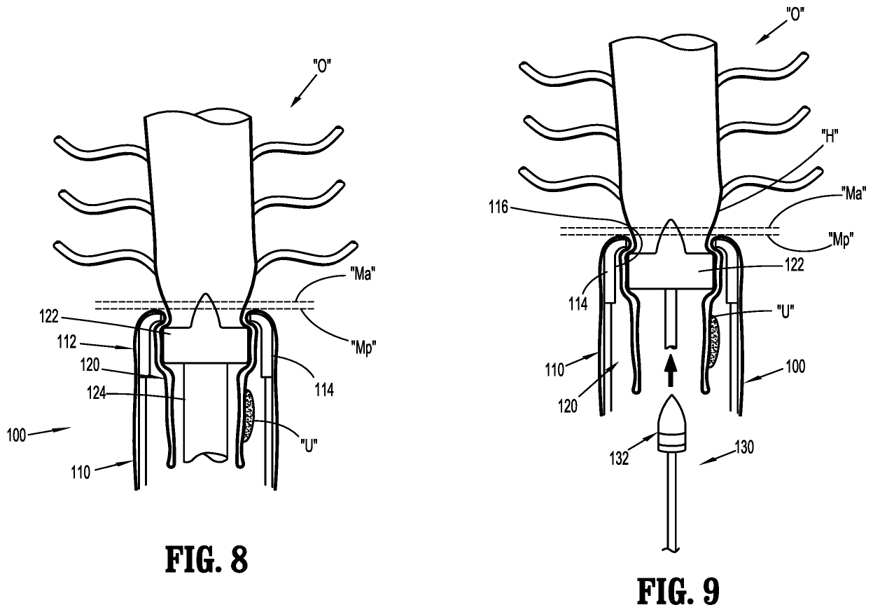

With reference now to FIG. 8, once all of the diseased tissue “U” is pulled into the tubular body 110 of the surgical stapling device 100, the respective proximal and distal margin lines “Mp”, “Md” are aligned with the tapered distal end portion 112 of the tubular body 110 of the surgical stapling device 100. In this manner, the healthy tissue “H” is aligned with the cylindrical anvil member 114 in the tapered distal end portion 112. The surgeon then inserts the cartridge assembly 120 through the tubular body 110 to align the staple cartridge 122 with the cylindrical inner surface 116 of the cylindrical anvil member 114 such that the healthy tissue “H” adjacent the proximal and distal margin lines “Mp”, “Md” is disposed between the staple cartridge 122 of the cartridge assembly 120 and the cylindrical inner surface 116 of the cylindrical anvil member 116 of the tubular body 110.

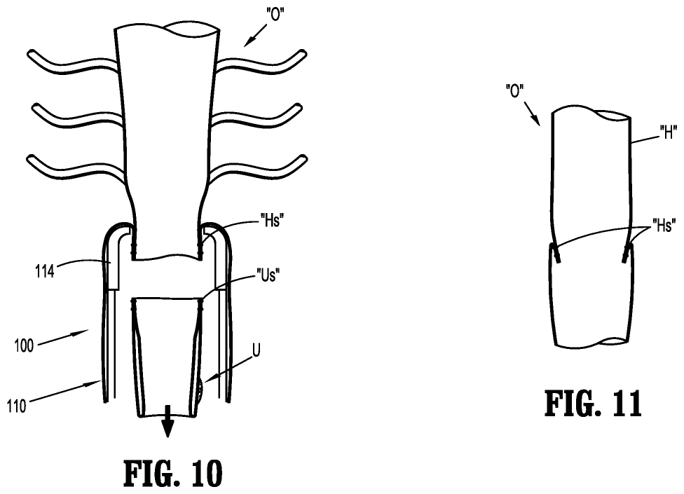

Turning to FIG. 9, the staple driving member 132 of the staple driver 130 is then directed through the staple cartridge 122 of the cartridge assembly 120 to cause the staples (not shown) to be ejected from within the staple cartridge 120 into the staple forming pockets 117 in the staple forming surface 116 of the cylindrical anvil member 114 of the tubular body 110. Although not shown, it is envisioned that the staple cartridge 120 includes a radially expandable cutting member that radially expands as the staple driving member 132 of the staple driver 130 is received through the staple cartridge 120 to cut the diseased tissue “U” from the health tissue “H” of the tubular organ “O” between the resulting staple line “Hs” (FIG. 10) of the healthy tissue “H” and the staple line “Ds” (FIG. 10) of the diseased tissue “U”.

Retraction of the staple driver 130 through the staple cartridge 122 of the cartridge assembly 120 permits the cartridge assembly 122 to return to its pre-actuated condition, thereby permitting removal of the cartridge assembly 120.

Turning to FIG. 10, once the diseased tissue “U” is separated from the healthy tissue “H”, the diseased tissue “U” may be removed from the patient through the tubular body 110 of the surgical stapling device 100. In this manner, the diseased tissue “U” is contained within the surgical stapling device 100 and does not come into contact with any additional healthy tissue “H” as the diseased tissue “U” is removed from the patient.

As shown in FIG. 11, after removal of the surgical stapling device 100 from within the tubular organ “O”, only health tissue “H” remains.

Although shown and described as using a surgical stapler, it is envisioned that the tissue removal procedure of the present disclosure may be modified for use with other instruments suitable for completing anastomosis procedures, e.g., suturing instrument instruments.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

1. A method for removing diseased tissue from a tubular organ, the method comprising:
receiving a distal end portion of a tubular body of a surgical stapling device within a tubular organ of a patient;
retracting a portion of the tubular organ including a diseased section of tissue within a lumen of the tubular body;
positioning proximal and distal margin lines of the portion of the tubular organ including the diseased section of tissue adjacent a cylindrical anvil member disposed on the distal end portion of the tubular body;
receiving a cartridge assembly within the tubular body in alignment with the cylindrical anvil member such that sections of healthy tissue are disposed between the cartridge assembly and the cylindrical anvil assembly;
ejecting a plurality of staples radially outward from within the cartridge assembly through the sections of healthy tissue disposed between the cartridge assembly and the cylindrical anvil member, and into the cylindrical anvil member; and
removing the diseased tissue through the tubular body of the surgical stapling device.

2. The method of claim 1, further including identifying the proximal and distal margin lines of the tubular organ.

3. The method of claim 1, further including ligating and severing blood vessels and connective tissue attached to the diseased section of tissue.

4. The method of claim 3, wherein ligating and severing the blood vessels and connective tissue includes using an electrocautery device.

5. The method of claim 1, wherein ejecting the plurality of staples further includes cutting the tissue adjacent the proximal and distal margin lines to separate the diseased section of tissue from the tubular organ.

6. The method of claim 1, wherein ejecting the plurality of staples includes receiving a drive member within the cartridge assembly.

7. The method of claim 1, wherein ejecting the plurality of staples includes forming first and second staple lines in the section of healthy tissue.

8. The method of claim 7, wherein ejecting the plurality of staples further includes cutting the section of healthy tissue between the first and second staple lines.

9. The method of claim 1, wherein retracting the portion of the tubular organ including a diseased section of tissue within the lumen of the tubular body includes inverting the portion of the tubular organ.

10. The method of claim 9, wherein inverting the portion of the tubular organ includes grasping tissue of the tubular organ distal of the section of diseased tissue and pulling the tissue proximally within the tubular body.
